(19) United States
(12) Patent Application Publication
(10) Pub. No.: US 2023/0165462 A1
(43) Pub. Date: Jun. 1, 2023

(54) FUNDUS PHOTOGRAPHING APPARATUS

(71) Applicant: NIDEK CO., LTD., Gamagori-shi (JP)

(72) Inventors: Shinya IWATA, Gamagori-shi (JP); Masayuki YOSHINO, Gamagori-shi (JP)

(73) Assignee: NIDEK CO., LTD., Gamagori-shi (JP)

(21) Appl. No.: 18/070,539

(22) Filed: Nov. 29, 2022

(30) Foreign Application Priority Data
Dec. 1, 2021 (JP) 2021-195593
Dec. 3, 2021 (JP) 2021-197217

(51) Int. Cl. A61B 3/12; A61B 3/117; A61B 3/10; A61B 3/15

(52) U.S. Cl. CPC A61B 3/12; A61B 3/1176; A61B 3/102; A61B 3/152

(57) ABSTRACT

A fundus photographing apparatus includes: a photographing unit including a front photographing optical system that forms, on a pupil of an examinee's eye, an illuminating light projection region and an illuminating light receiving region next to each other in a first direction, the front photographing optical system scanning a fundus of examinee's eye with illuminating light to acquire two-dimensional reflection image of the fundus; a driver that moves the photographing unit relative to examinee's eye; and a processor that switches, between a first and a second alignment mode, a control of guiding a positional relation between the examinee's eye and the photographing unit, the positional relation being guided to a first alignment state in predetermined positional relationship in the first alignment mode, and being guided to a second alignment state displaced at least in a direction crossing the first direction from the first alignment state in the second alignment mode.

### CROSS-REFERENCE TO RELATED APPLICATION

This application claims priority from Japanese Patent Application Nos. 2021-195593 filed on Dec. 1, 2021, and 2021-197217 filed on Dec. 3, 2021, with the Japan Patent Office, the entire contents of which are hereby incorporated by reference.

### BACKGROUND ART

### 1. Technical Field

The present disclosure relates to a fundus photographing apparatus.

### 2. Related Art

A fundus photographing apparatus such as a fundus camera or an OCT device has been widely utilized in an ophthalmic field. In a case where there is opacity on the optic media of an examinee's eye, the fundus cannot be favorably photographed in some cases because projection and reception of photographing light by the fundus photographing apparatus are interfered by the opacity.

In response, a technique of performing alignment adjustment so as to avoid the opacity to acquire a fundus image in the case of photographing the fundus of the examinee's eye with the opaque optic media has been known.

For example, in an apparatus described in JP-A-2018-186930, three-dimensional OCT data on the anterior segment is obtained by photographing, and in this manner, opacity distribution is obtained. Thereafter, alignment adjustment for fundus photographing is performed so as to avoid opacity.

As a technique of acquiring, as an image, opacity distribution on the optic media, a retro-illumination method has been known. The retro-illumination method is a technique of observing light reflected from the fundus after the pupil has been irradiated with such light. In an apparatus described in JP-A-2017-99718, a dedicated light source is provided to acquire a retro-illumination image by use of an anterior segment observing optical system.

JP-A-2017-99718 discloses an optical system of a fundus camera which is currently in widespread use in many ophthalmic facilities. The fundus camera simultaneously irradiates an entire photographing area of the fundus with photographing light, and based on fundus reflection light, photographs a two-dimensional reflection image of the fundus. Generally, in the fundus camera, pupil division is set such that the periphery of the pupil of an examinee's eye is irradiated in a ring shape with the photographing light and the fundus reflection light is taken out from the pupil center.

### SUMMARY

A fundus photographing apparatus includes: a photographing unit including a front photographing optical system that forms, on a pupil of an examinee's eye, an illuminating light projection region and an illuminating light receiving region next to each other in a first direction, the front photographing optical system scanning a fundus of the examinee's eye with illuminating light to acquire a two-dimensional reflection image of the fundus; a driver that moves the photographing unit relative to the examinee's eye; and a processor that switches, between a first alignment mode and a second alignment mode, a control of guiding a positional relation between the examinee's eye and the photographing unit, the positional relation being guided to a first alignment state in a predetermined positional relationship in the first alignment mode, and being guided to a second alignment state displaced at least in a direction crossing the first direction from the first alignment state in the second alignment mode.

### BRIEF DESCRIPTION OF DRAWINGS

FIG. 1 is an external view of a fundus photographing apparatus;

FIG. 2 is a schematic diagram of optical systems of the fundus photographing apparatus;

FIG. 3 is a schematic diagram of a front photographing optical system;

FIG. 4A is a view showing an example of a fundus observation image on which split targets are projected, and shows an image in a case where a focus state is not properly adjusted;

FIG. 4B is a view showing an example of the fundus observation image on which the split targets are projected, and shows an image in a case where the focus state is properly adjusted;

FIG. 5 is a schematic diagram of an anterior segment observing optical system;

FIG. 6 is a schematic diagram of an OCT optical system;

FIG. 7 is a block diagram showing a control system of the fundus photographing apparatus;

FIG. 8 is a flowchart showing the flow of operation of an apparatus in an example;

FIG. 9 is a view showing an anterior segment observation image;

FIG. 10A is a view for describing a small-pupil mode, and shows an anterior segment observation image in a state in which light projected at a certain alignment reference position via each of light projection regions P1, P2 is eclipsed by iris;

FIG. 10B is a view for describing the small-pupil mode, and shows an anterior segment observation image in a state in which the alignment reference position is offset in an X-direction from FIG. 10A;

FIG. 11 is a flowchart showing second alignment adjustment;

FIG. 12A is a view for describing the second alignment adjustment for a cataract eye in a normal state;

FIG. 12B is a view for describing the second alignment adjustment in the normal state, and is different from FIG. 12A in a positional relation between an examinee's eye and a photographing unit;

FIG. 13A is a view for describing the second alignment adjustment in the small-pupil mode;

FIG. 13B is a view for describing the second alignment adjustment in the small-pupil mode, and is different from FIG. 13A in the positional relation between the examinee's eye and the photographing unit; and

FIG. 14 is a view for describing a modification.

### DETAILED DESCRIPTION

In the following detailed description, for purpose of explanation, numerous specific details are set forth in order to provide a thorough understanding of the disclosed embodiments. It will be apparent, however, that one or more embodiments may be practiced without these specific details. In other instances, well-known structures and devices are schematically shown in order to simplify the drawing.

In the technique of JP-A-2018-186930 as a technique of checking the presence or absence of the opacity of the examinee's eye or the degree of the opacity of the examinee's eye, upon photographing of the fundus, the three-dimensional OCT data on the anterior segment needs to be obtained by photographing after an OCT optical system has been adjusted relative to the anterior segment once in advance. For this reason, it is difficult to smoothly acquire the fundus image. For utilizing the retro-illumination method, an apparatus configuration tends to be complicated, such as use of the dedicated light source.

In the fundus camera optical system disclosed in JP-A-2017-99718, in a case where alignment adjustment to a position apart from the center (e.g., the corneal apex) of the examinee's eye is performed so as to avoid the opacity, flare due to reflection light from the optic media is easily caused.

One object of the present disclosure is to provide a fundus photographing apparatus capable of favorably photographing the fundus of an examinee's eye with the opaque optic media.

A fundus photographing apparatus according to a first aspect of the present disclosure includes: a photographing unit including a front photographing optical system that forms, on a pupil of an examinee's eye, an illuminating light projection region and an illuminating light receiving region next to each other in a first direction, the front photographing optical system scanning a fundus of the examinee's eye with illuminating light to acquire a two-dimensional reflection image of the fundus; a driver that moves the photographing unit relative to the examinee's eye; and a processor that switches, between a first alignment mode and a second alignment mode, a control of guiding a positional relation between the examinee's eye and the photographing unit, the positional relation being guided to a first alignment state in a predetermined positional relationship in the first alignment mode, and being guided to a second alignment state displaced at least in a direction crossing the first direction from the first alignment state in the second alignment mode.

### SUMMARY

Embodiments of a fundus photographing apparatus according to the present disclosure will be described. Each embodiment is applicable to part or the entirety of other embodiments. For example, items below in angle brackets may be utilized independently of or in association with each other.

The fundus photographing apparatus according to each embodiment of the present disclosure performs alignment adjustment for an examinee's eye with opaque optic media such that influence of the opacity is reduced, thereby acquiring a fundus photographic image.

The fundus photographing apparatus according to each embodiment of the present disclosure has at least a photographing unit, a driver, and a controller (a processor).

### First Embodiment

First, a fundus photographing apparatus according to a first embodiment will be described.

### <Photographing Unit>

A photographing unit in the present embodiment includes at least a photographing optical system and an observing optical system. The photographing unit may include various optical systems of the fundus photographing apparatus. For example, the photographing unit may include at least any of an anterior segment observing optical system and a target projecting optical system. The driver may integrally change a positional relation between each optical system and an examinee's eye.

### <Photographing Optical System>

The photographing optical system is utilized for projecting photographing light onto the examinee's eye and receiving the photographing light from the examinee's eye to acquire a fundus photographic image.

### <Front Photographing Optical System>

The photographing optical system may be, for example, a front photographing optical system. The front photographing optical system acquires, as a photographic image, a two-dimensional reflection image of the fundus based on the photographing light reflected from the fundus. The photographing optical system may be a scanning optical system or a non-scanning optical system. Examples of the scanning optical system include a spot scan type optical system and a line scan type optical system. In the spot scan type optical system, the fundus is two-dimensionally scanned with point-shaped photographing light. In the line scan type optical system, the fundus is scanned in one direction with line-shaped photographing light (details will be described later in a second embodiment). Examples of the non-scanning optical system include an optical system of a general fundus camera.

### <OCT Optical System>

The photographing optical system may be, for example, an OCT optical system. The OCT optical system is utilized for obtaining, by photographing, OCT data on the fundus based on a principle (specifically, an optical interference principle) different from that of the front photographing optical system. That is, the OCT optical system detects a spectral interference signal of reference light and measurement light guided to the fundus of the examinee's eye. In this case, the measurement light is used as the photographing light. The spectral interference signal is processed, and in this manner, the OCT data on the fundus is obtained by imaging.

The OCT optical system has at least an OCT light source and a detector. The detector detects the state of interference of the measurement light and the reference light emitted from the OCT light source. The OCT optical system may additionally have an optical scanner for scanning the fundus with the measurement light. A basic configuration of the OCT optical system may be a Fourier-domain OCT optical system. For example, the OCT optical system may be a spectral-domain OCT (SD-OCT) optical system or a swept-source OCT (SS-OCT) optical system. Alternatively, the OCT optical system may be a time-domain OCT (TD-OCT) optical system.

The fundus photographing apparatus may include, as the photographing optical system, both the front photographing optical system and the OCT optical system.

### <Observing Optical System>

The observing optical system (also referred to as a fundus observing optical system) is utilized for projecting observation light, which is different from the photographing light and is infrared light, onto the fundus of the examinee's eye and receiving the observation light from the fundus to acquire a fundus observation image which is a two-dimensional reflection image based on the observation light. The observation light and the photographing light may be different from each other in, e.g., a wavelength. The fundus observation image is acquired as needed substantially in real time. The fundus observation image is, for example, utilized for adjusting various photographing conditions. Moreover, in the present embodiment, the fundus observation image is utilized for adjusting (and guiding) a positional relation between the examinee's eye and the photographing unit (details will be described later). As in the above-described front photographing optical system, the observing optical system may be a scanning optical system or a non-scanning optical system.

In a case where the photographing optical system is the front photographing optical system, some or all of members may be shared by the photographing optical system and the observing optical system.

### <Target Projecting Optical System>

The target projecting optical system projects, onto the fundus, a target light flux for adjusting the photographing conditions for the photographing optical system. The target light flux is imaged by the observing optical system. As a result, a target image is on the observation image. The controller adjusts the photographing conditions based on the target image. Further, the target image may be utilized for adjusting the positional relation between the examinee's eye and the photographing unit (details will be described later).

The target projecting optical system may project a target light flux for focus adjustment, for example. The target light flux for focus adjustment may be a split target. In this case, at least two target light fluxes forming split targets pass through different positions of the anterior segment of the examinee's eye, and then, are projected onto the fundus. A focus state is detected as the state of separation and matching of the split targets. Based on the focus state, diopter correction is performed in the photographing optical system and the observing optical system.

### <Anterior Segment Observing Optical System>

The fundus photographing apparatus of the first embodiment may have the anterior segment observing optical system, and an anterior segment observation image may be acquired via the anterior segment observing optical system. The anterior segment observing optical system may have at least an imaging element. The anterior segment observation image is utilized for adjusting the positional relation between the examinee's eye and the photographing unit (i.e., alignment, tracking, and the like). The anterior segment observation image may be, for example, a front image of the anterior segment or an image of the anterior segment from a diagonal direction. Upon alignment, the anterior segment observation image may be displayed on a monitor. Accordingly, an examiner can grasp a real-time alignment state.

The anterior segment observation image may be captured by an imaging element different from the imaging element or the detector of the photographing optical system. In addition to the imaging element, the anterior segment observing optical system may include various optical elements such as a light source.

Further, the fundus photographing apparatus may have an alignment target projecting optical system that projects an alignment target onto the examinee's eye. Based on the alignment target on the observation image of the anterior segment or the fundus, alignment may be guided.

### <Driver>

The driver is a mechanism that moves the photographing unit relative to the examinee's eye. In this case, the driver can change the position of the photographing unit relative to the examinee's eye at least in X- and Y-directions (up-down and right-left directions). More preferably, the driver can also change the position of the photographing unit relative to the examinee's eye in a Z-direction. The driver is used for adjusting the positional relation between the examinee's eye and the photographing unit.

The driver may have an actuator that changes the positional relation between the examinee's eye and the photographing unit based on a signal from the controller. The driver may displace the photographing unit, displace a face supporting unit (e.g., a chin rest) that supports the face of an examinee, or displace both thereof.

### <Controller>

The controller is the processor that controls various types of control operation and calculation in the fundus photographing apparatus.

For example, the controller performs alignment control. In the first embodiment, the controller guides the positional relation between the examinee's eye and the photographing unit at least in the X- and Y-directions based on the fundus observation image. In this case, the positional relation between the examinee's eye and the photographing unit may be guided to such a positional relation that the optical axis of the photographing unit is not coincident with the center (e.g., the pupil center or the corneal apex) of the anterior segment. Note that the alignment control based on the fundus observation image according to the first embodiment may be executed after transition to an alignment state in which the fundus observation image can be acquired.

In the present embodiment, an alignment guide method may be a so-called automatic alignment method or a manual alignment method. In the automatic alignment method, the controller may drivably control the driver based on the fundus observation image. In the manual alignment method, the controller may cause the monitor to display the anterior segment observation image and to display, based on the fundus observation image, a guide (e.g., an electronic reticle) for guiding operation to a target position. In this case, the fundus photographing apparatus may include an operation inputter. The operation inputter is used for driving, in response to examiner's operation, the driver according to such operation to adjust the positional relation between the examinee's eye and the photographing unit. The operation inputter may be an input interface for transmitting an operation of driving the actuator of the driver or a mechanical component directly acting on the driver.

For example, in a case where the opacity is caused on the optic media of the examinee's eye and at least part of the projected and received observation light is blocked due to the opacity, the observation image is influenced by the opacity. For example, due to the influence of the opacity, brightness degradation, uneven brightness, contrast degradation, and shade caused by an eclipse by iris due to the opacity, and the like are caused on the observation image. By evaluation of the influence of the opacity, on the observation image, the presence or absence of the opacity or the degree of opacity at a position, which is targeted for projection and reception of the observation light, on the pupil of the examinee's eye in the positional relation between the examinee's eye and the photographing unit at the time can be estimated. In this case, evaluation may be performed based at least on any of the brightness and contrast of the observation image. Specifically, the influence of the opacity on the observation image may be evaluated based on the degree of degradation of any of the brightness and contrast of the observation image.

Note that an evaluation method is not limited to above. For example, in a case where the observation image is acquired in a state in which the target light flux from the target projecting optical system is projected on the fundus, the influence of the opacity on the observation image may be evaluated based on a target image detection result. In this case, the presence or absence of the opacity or the degree of opacity at a position, which is targeted for projection and reception of the target light flux, on the pupil of the examinee's eye is estimated based on the target image detection result. The influence of the opacity on the observation image may be evaluated based at least on any of the presence or absence of the target image, the brightness of the target image, and the contrast of the target image.

Note that in a case where the controller evaluates the influence of the opacity on the observation image, evaluation may be performed based on a partial region on the observation image. For example, evaluation may be preferably performed based on a center portion of the observation image. The center portion of the image is less susceptible to influence of uneven brightness even in a case where the optical axis of the photographing unit has displaced from the center of the anterior segment. For this reason, the center portion of the image is used so that the influence of the opacity on the observation image can be easily properly evaluated. Even if flare due to the optic media is caused, the center portion of the observation image is less susceptible to influence of such flare. Thus, the center portion of the image is used so that the influence of the opacity on the observation image can be easily properly evaluated.

In the first embodiment, the positional relation between the examinee's eye and the photographing unit is guided at least in the X- and Y-directions based on the fundus observation image. Thus, even in, e.g., a case where the examinee's eye with the opaque optic media is photographed, the above-described positional relation can be guided based on the fundus observation image such that the influence of the opacity is reduced, and the alignment state can be adjusted accordingly. That is, in a case where the examinee's eye with the opaque optic media is photographed, the controller may guide the positional relation between the examinee's eye and the photographing unit so as to avoid the opacity.

In a case where the positional relation between the examinee's eye and the photographing unit is guided based on the fundus observation image, the controller may search such a positional relation between the examinee's eye and the photographing unit that a better observation image can be acquired. In this case, the controller may acquire multiple fundus observation images in multiple different positional relations at least in the X- and Y-directions. Further, the controller may guide the positional relation between the examinee's eye and the photographing unit based on these multiple fundus observation images. In this case, the controller can guide the positional relation between the examinee's eye and the photographing unit such that the influence of the opacity is more effectively reduced. For example, the multiple fundus observation images may be acquired in association with the alignment state (the positional relation between the examinee's eye and the photographing unit) at the time. After the multiple fundus observation images in the multiple positional relations between the examinee's eye and the photographing unit have been acquired, the positional relation between the examinee's eye and the photographing unit may be guided again to such a positional relation that the influence of the opacity is more suitably reduced, and then, photographing may be executed. Note that upon searching, the multiple positional relations in which the multiple fundus observation images are acquired may transition (be selected) in a preset pattern or a random pattern. Moreover, a timing of terminating searching can be set as necessary. For example, searching may be completed when the observation image acquired as needed satisfies a predetermined condition.

In the first embodiment, since the positional relation between the examinee's eye and the photographing unit is guided based on the fundus observation image, an apparatus configuration is less complicated. In the apparatus including the OCT optical system, a case where alignment is performed so as to avoid the opacity based on the OCT data acquired via the OCT optical system is also assumed. As compared to such a case, a flow from alignment to photographing is assumed to be smoother in a case where the positional relation between the examinee's eye and the photographing unit is guided based on the fundus observation image. That is, a time required for various types of adjustment until photographing is more easily shortened in the fundus observing optical system than in the OCT optical system. Generally, a region, which is targeted for light projection and reception, on the pupil of the examinee's eye is wider in the observing optical system than in the OCT optical system. Thus, the efficiency of searching the positional relation between the examinee's eye and the photographing unit is better. Further, as compared to the case of utilizing the OCT data, the case of guiding the positional relation between the examinee's eye and the photographing unit based on the fundus observation image is more suitable for acquiring not only the OCT data but also the two-dimensional reflection image as the photographic image.

Although details will be described later in the second embodiment, in a case where the photographing optical system acquires the two-dimensional reflection image as the photographic image, the photographing optical system is preferably the scan type optical system. In a case where the photographing optical system is the scan type optical system, the photographic image with reduced flare due to the optic media can be easily acquired even if photographing is performed with the optical axis of the photographing unit displaced from the center of the anterior segment as a result of the positional relation between the examinee's eye and the photographing unit being guided so as to avoid the opacity of the optic media.

The controller may acquire pupil information on the examinee's eye. The pupil information may be information regarding a pupil region, and for example, may be information for specifying at least any of the position, shape, size, and the like of the pupil of the examinee's eye. The pupil information is, for example, acquired based on the anterior segment observation image. In a case where the size of the pupil (e.g., a pupil diameter) is acquired as the pupil information, the pupil information may be acquired from an external apparatus or based on examiner's operation input. In this case, the fundus photographing apparatus may have an input interface.

The controller may guide the positional relation between the examinee's eye and the photographing unit, considering the pupil information. Note that the pupil information may be acquired in real time based on the anterior segment observation image. Further, the real-time pupil information may be utilized for a control of guiding the positional relation.

An area, which is an effective movement area for searching such a positional relation that the observation light is favorably projected onto and received from the fundus, where the relative positions of the examinee's eye and the photographing unit are moved in the X- and Y-directions can be specified based on the pupil information. Thus, the pupil information is taken into consideration so that alignment adjustment based on the observation image can be more smoothly performed.

In a case where the information regarding the pupil size of the examinee's eye is acquired as the pupil information, the controller may change, according to the pupil size, a control of guiding the positional relation between the examinee's eye and the photographing unit based on the fundus observation image. For example, at least any of the movement area, a movement direction, a movement pattern, and the like when the relative positions of the examinee's eye and the photographing unit are changed based on the observation image may be differentiated among different pupil sizes.

For example, in some cases, pupil division is set for the photographing optical system and the observing optical system such that an observation light projection region and an observation light receiving region on the pupil of the examinee's eye are formed next to each other in one direction. In the case of an eye with a small pupil, when the relative positions of the examinee's eye and the photographing unit are moved in the above-described one direction, the photographing light and the observation light are easily eclipsed by iris. In this case, in a case where the pupil size is larger than a threshold, the direction of movement when the relative positions of the examinee's eye and the photographing unit are changed based on the observation image is not necessarily limited. In a case where the pupil size is the threshold or less, the direction of movement when the relative positions of the examinee's eye and the photographing unit are changed based on the observation image may be limited to a direction crossing the one direction. In this case, proper adjustment to the alignment state in which the influence of the opacity is reduced is easily performed.

Generally, in the OCT optical system, a required pupil diameter is smaller than that of the observing optical system. For this reason, in, e.g., a case where the photographing optical system is the OCT optical system, if the pupil size is larger than the threshold, the relative positions of the examinee's eye and the photographing unit may be changed based on the observation image, in a case where the pupil size is the threshold or less, the relative positions of the examinee's eye and the photographing unit may be changed based on the OCT data acquired via the OCT optical system.

### <Reutilization of Search Result>

In a case where the controller searches the positional relation between the examinee's eye and the photographing unit and photographing is performed, a result of searching for photographing may be reutilized thereafter. For example, this result may be utilized for alignment guide upon follow-up photographing. In a case where a position presented for, e.g., fixation is changed between a first position and a second position and photographing is performed thereafter, alignment guide at the second position may be performed based on a result of search conducted at the first position. According to the position presented for, e.g., fixation, a positional relation between the optical axis of the photographing unit and the pupil of the examinee's eye changes. For this reason, the result of search conducted at the first position may be saved with information on the position of the pupil of the examinee's eye at the time. For example, a result of adjustment of the positional relation between the examinee's eye and the photographing unit at the first position may be saved with reference to the pupil center position. The positional relation between the examinee's eye and the photographing unit may be guided based on the position of the pupil acquired from the anterior segment observation image upon alignment adjustment at the second position and the above-described adjustment result acquired in advance at the first position. Note that the first position may be, for example, such a position presented for, fixation that the photographic image can be acquired about the macula. The second position may be such a position presented for, e.g., fixation that the photographic image can be acquired about the optic disc (or about an intermediate point between the macula and the optic disc).

### <Condition for Starting Alignment Control Based on Fundus Observation Image>

In the fundus photographing apparatus of the first embodiment, it may be switchable whether or not the alignment control based on the fundus observation image is to be started.

For example, according to examiner's operation input, it may be switchable in advance whether or not the alignment control based on the fundus observation image is to be executed.

For example, after adjustment for obtaining the alignment state in which the fundus observation image can be acquired has been performed as necessary, the controller estimates the influence of the opacity based on the fundus observation image. In this manner, it may be determined whether or not the above-described alignment control is to be started. According to a determination result, the alignment control based on the fundus observation image may be automatically started. Alternatively, according to the determination result, the controller may request, via a user interface, the examiner to issue an instruction for starting the alignment control based on the fundus observation image, for example.

For example, in a case where information indicating that the examinee's eye is a cataract eye is associated with an ID for an examinee in a previous test, the alignment control based on the fundus observation image may be automatically started according to the information associated with the ID for the examinee. Alternatively, the controller may request, via the user interface, the examiner to issue the instruction for starting the alignment control based on the fundus observation image, for example. In a case where the examinee's eye is associated with information indicating that the examinee's eye is an IOL-inserted eye, there is a high probability of no opacity problem being caused. Thus, alignment adjustment may be completed without the alignment control based on the fundus Observation image.

### Second Embodiment

Next, the second embodiment of the present disclosure will be described.

### <Scanning Front Photographing Optical System>

In the second embodiment, a photographing unit has at least a scanning front photographing optical system. The front photographing optical system may be a spot scan type optical system or a line scan type optical system. In description below, the front photographing optical system in the second embodiment also serves as an observing optical system, unless otherwise specified. For the sake of convenience in description, photographing light and observation light will be collectively referred to as “illuminating light.”

In the second embodiment, the photographing unit may include the front photographing optical system that forms, on the pupil of an examinee's eye, an illuminating light projection region and an illuminating light receiving region next to each other in a first direction and scans the fundus of the examinee's eye with the illuminating light to acquire a two-dimensional reflection image of the fundus.

In the second embodiment, the front photographing optical system has at least an irradiation optical system, a light receiving optical system, a scanner, and a harmful light remover.

The irradiation optical system irradiates the fundus of the examinee's eye with the illuminating light via an objective optical system. Additionally, the irradiation optical system may have a light source (an illuminating light source) that emits the illuminating light. The light receiving optical system has a light receiving element that receives the illuminating light reflected from the fundus. A signal from the light receiving element is input to an image processor. In the image processor, the two-dimensional reflection image of the fundus of the examinee's eye is acquired based on the signal from the light receiving element. Note that as the light receiving element, any of a point light receiving element, a line sensor, a two-dimensional light receiving element (a photographing elements, and the like may be employed as necessary according to an optical system.

Some optical elements may be shared by the irradiation optical system and the light receiving optical system. For example, the objective optical system and an optical path coupler may be shared. The optical path coupler couples and separates an illuminating light projection optical path and a fundus reflection light receiving optical path. In this case, the objective optical system is arranged on an optical path common between the light projection optical path and the light receiving optical path formed by the optical path coupler.

In the second embodiment, the irradiation optical system forms a local illuminating region on part of a photographing area of the fundus. That is, the irradiation optical system irradiates the fundus locally with the illuminating light. The irradiation optical system forms, as a typical example, a slit-shaped or point-shaped illuminating region.

The harmful light remover may be arranged at a position conjugated with the fundus on the optical path of the light receiving optical system.

The harmful light remover causes the light receiving element to receive the fundus reflection light from a local photographing region (hereinafter referred to as an “effective region”) which is part of the photographing area. Moreover, the harmful light remover removes light from a location other than the effective region. The harmful light remover may be, for example, a diaphragm. Examples of a typical diaphragm in the spot scan apparatus include a pinhole. Examples of a typical diaphragm in the slit scan apparatus include a slit. In this case, the fundus reflection light from the effective region, which corresponds to an opening of the diaphragm, of the entire photographing area of the fundus is selectively guided to the light receiving element, and is acquired as an effective image. Specifically, in the slit scan apparatus, the light receiving element also serves as the harmful light remover in some cases. In this case, as the light receiving element, a line sensor in the form of a slit may be used, or a CMOS configured such that line exposure is performed on a two-dimensional imaging surface (in other words, a CMOS having a rolling shutter function) may be used. In this case, the fundus reflection light from the effective region, which corresponds to line-shaped effective pixels, of the entire photographing area of the fundus is selectively guided to the light receiving element, and the effective region is imaged accordingly.

The scanner scans, in synchronization, the local illuminating region of the fundus and the effective region (the local photographing region) of the fundus. The scanner may be, for example, an optical scanner shared by the irradiation optical system and the light receiving optical system. In this case, the optical scanner is arranged on the optical path common between the irradiation optical system and the light receiving optical system.

The scanner may include a first scanner provided in the irradiation optical system and a second scanner provided in the light receiving optical system, the second scanner being different from the first scanner. In this case, in one example of the slit scan apparatus, a first slit-shaped member for forming the local illuminating region into a slit shape may be arranged on the optical path of the irradiation optical system. The first scanner may have the first slit-shaped member and a driver that moves the first slit-shaped member in a direction crossing the optical axis. In a case where a second slit-shaped member is used as the harmful light remover, the second scanner may have the second slit-shaped member and a driver that moves the second slit-shaped member in a direction crossing the optical axis. The driver of the first scanner and the driver of the second scanner may be different devices or the same device.

In a case where the CMOS is used as the light receiving element in the slit scan apparatus, the CMOS may also serve as the second scanner. That is, line exposure by the above-described rolling shutter function may be controlled in synchronization with the first scanner. In this case, the CMOS as the light receiving element serves as both the harmful light remover and the second scanner. With this configuration, the number of components of the optical system is reduced.

Note that in the line scan type optical system, scan with line-shaped illuminating light in one direction is performed. For example, the fundus may be linearly scanned with the line-shaped illuminating light, or may be rotatably scanned with the line-shaped illuminating light. In the case of the rotary scan, the center of rotation may be the optical axis of the front photographing optical system.

### <Form of Pupil Division in Front Photographing Optical System>

Of a pupil image formed on the pupil of the examinee's eye in the front photographing optical system, a region through which light passes from the apparatus to the fundus will be referred to as a light projection region and a region through which the fundus reflection light passes will be referred to as a light receiving region, in the present embodiment.

In the front photographing optical system of the second embodiment, at least the light projection region and the light receiving region next to each other in the first direction are formed on the pupil of the examinee's eye. The light projection region and the light receiving region next to each other in the first direction are non-concentrically arranged in line. That is, each of the light projection region and the light receiving region is arranged such that one of the light projection region or the light receiving region is not surrounded by the other one of the light projection region or the light receiving region with no clearance as in a general fundus camera optical system in which pupil division is set by a ring slit and a hall mirror. Alternatively, the light projection region and the light receiving region may be arranged on the pupil of the examinee's eye such that one of the light projection region or the light receiving region is not arranged in a direction crossing the first direction with respect to the other one of the light projection region or the light receiving region.

At least any of the light projection region and the light receiving region may include multiple regions formed at different positions in the first direction. In order to reduce flare due to the optic media, the first direction is, in the slit scan apparatus, preferably coincident with the direction of scanning the fundus with the illuminating light.

### <Mode Switching in Alignment Control>

In the second embodiment, a controller switches, between a first alignment mode and a second alignment mode, a control of guiding a positional relation between the examinee's eye and the photographing unit (the front photographing optical system). Determination on whether the first alignment mode or the second alignment mode is to be set may be made based on information (hereinafter referred to as examinee's eye opacity information) regarding opacity in the optic media of the examinee's eye, for example. Alternatively, determination on whether the first alignment mode or the second alignment mode is to be set may be made based on examiner's mode switching operation.

In the first alignment mode, the positional relation between the examinee's eye and the photographing unit is guided to a first alignment state in a predetermined positional relation. In the second alignment mode, the positional relation between the examinee's eye and the photographing unit is guided to a second alignment state displaced from the first alignment state. In the second embodiment, the single light projection region and the single light receiving region are next to each other in the first direction on the pupil of the examinee's eye. Thus, there is a small margin (space) for moving the light projection region and the light receiving region in the first direction within a pupil region. For this reason, the opacity is less likely to be avoided only by movement of the light projection region and the light receiving region in the first direction within the pupil region.

On the other hand, in the second embodiment, the controller guides, in the case of the second alignment mode, the positional relation between the examinee's eye and the photographing unit to the second alignment state displaced at least in the direction crossing the first direction from the first alignment state. In the above-described front photographing optical system, the margin (space) for moving the light projection region and the light receiving region within the pupil region is easily ensured in the direction crossing the first direction. Thus, it is assumed that the opacity is easily avoided in such a manner that the positional relation is guided in the direction crossing the first direction.

Note that the direction crossing the first direction may be a direction perpendicular to the first direction or a direction diagonally crossing the first direction.

Even in a case where photographing is performed with the optical axis of the front photographing optical system displaced from the center of the anterior segment as a result of the positional relation between the examinee's eye and the photographing unit being guided so as to avoid the opacity of the optic media, the flare due to the optic media can be reduced by use of the above-described scanning optical system as the front photographing optical system. That is, in the scanning optical system, the entire photographing area of the fundus is irradiated locally with the illuminating light. Thus, in the scanning optical system, the projected and received light is favorably separated on the optic media as compared to a general fundus camera configured to simultaneously irradiate an entire photographing area with illuminating light, and the flare is less likely to be caused. As described above, in the second embodiment, the fundus of the examinee's eye with the opaque optic media is favorably photographed in the second alignment mode.

### <Acquisition of Opacity Information>

The controller may acquire opacity information on the examinee's eye. The opacity information may be information indicating at least any of the presence or absence of the opacity, the degree of opacity, or opacity distribution. The first embodiment describes that the presence or absence of the opacity or the degree of opacity at a certain position on the pupil of the examinee's eye can be estimated based on the fundus observation image. Thus, the opacity information may be acquired based on the fundus observation image. Note that in the second embodiment, the opacity information is not limited to such information. For example, the opacity information may be acquired based at least on any of a retro-illumination image, OCT data on the anterior segment, or OCT data on the fundus. Alternatively, the opacity information may be acquired based on a test result obtained in a previous test. In this case, an ID for an examinee and the opacity information may be stored in advance in a memory in association with each other.

As described above, the controller nay select, based on the opacity information, whether alignment control is to be executed in the first alignment mode or the second alignment mode.

The opacity information may be utilized in order to predict such a positional relation between the examinee's eye and the photographing unit that the opacity can be avoided in the second alignment mode or evaluate influence of the opacity.

As in the first embodiment, the controller may further acquire pupil information as information regarding the pupil region of the examinee's eye, and considering the pupil information, may guide the positional relation between the examinee's eye and the photographing unit. In this case, the controller may acquire information regarding the pupil size of the examinee's eye as the pupil information, and change a control of guiding the positional relation based on the fundus observation image according to the pupil size, for example.

In the second embodiment, the photographing unit may further include the above-described OCT optical system. The OCT optical system is utilized for obtaining, by photographing, the OCT data on the fundus based on an optical interference principle. In the second alignment mode, the controller may execute photographing (capturing) of the OCT data on the fundus and the two-dimensional reflection image of the fundus in the second alignment state. That is, after the positional relation between the examinee's eye and the photographing unit has been guided to the second alignment state in the second alignment mode, the controller may control the OCT optical system to acquire the OCT data on the fundus, and control the front photographing optical system to photograph the two-dimensional reflection image of the fundus. In this case, the two-dimensional reflection image of the fundus may be a color fundus image.

### First Example

One example of the fundus photographing apparatuses according to the first and second embodiments will be described.

A fundus photographing apparatus 1 photographs a color fundus image as a two-dimensional reflection image of a fundus Er. Further, the fundus photographing apparatus 1 obtains, by imaging, OCT data on an examinee's eye.

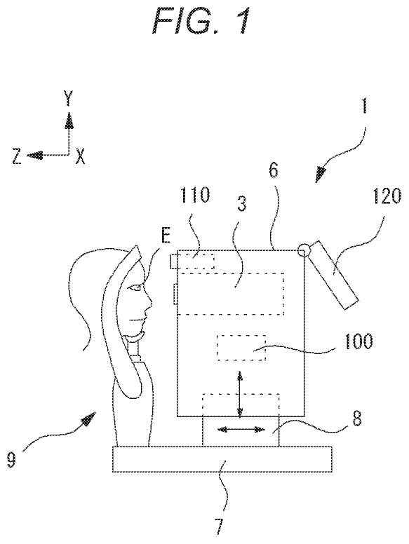

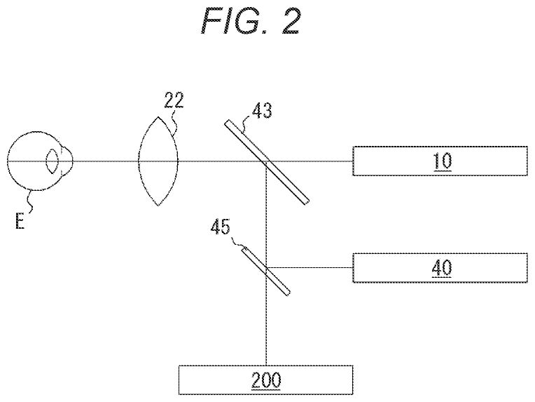

FIG. 1 is an external view of the fundus photographing apparatus 1. The fundus photographing apparatus 1 has a photographing unit 3. The photographing unit 3 mainly includes optical systems shown in FIG. 2. The fundus photographing apparatus 1 has a base 7, a driver 8, a face supporting unit 9, and a face photographing camera 110. Using these components, the fundus photographing apparatus 1 adjusts a positional relation between the examinee's eye E and the photographing unit 3.

The driver 8 moves the photographing unit 3 relative to the examinee's eye E. That is, the driver 8 moves the photographing unit 3 thereon relative to the examinee's eye E in each of X-, Y-, and Z-directions. The driver 8 has an actuator for moving the photographing unit 3 in each movement direction, and is driven based on a control signal from a controller 100. The face supporting unit 9 supports the face of an examinee. The face supporting unit 9 is fixed to the base 7.

The face photographing camera. 110 photographs the face of the examinee. The controller 100 specifies the position of the examinee's eye E from a photographed facial image and drivably controls the driver 8, thereby adjusting the position of the photographing unit 3 relative to the specified position of the examinee's eye E.

The photographing apparatus 1 further has a monitor 120. The monitor 120 displays various photographic images, observation images, and the like.

### <Photographing Unit>

FIG. 2 is a schematic diagram of the optical systems of the fundus photographing apparatus 1. In the present example, the fundus photographing apparatus 1 has a front photographing optical system 10, an anterior segment observing optical system 40, and an OCT optical system 200. In the present example, an objective lens 22 is shared by these optical systems. In the present example, the front photographing optical system 10 also serves as a fundus observing optical system. These optical systems are provided in the photographing unit 3.

In the present example, the optical axis of the anterior segment observing optical system 40 and the optical axis of the OCT optical system 200 are coaxial via a half mirror 45. Moreover, in the present example, the optical axes, which are coaxial via the half mirror 45, of the anterior segment observing optical system 40 and the OCT optical system 200 and the optical axis of the front photographing optical system 10 are coaxial via a dichroic mirror 43. For example, light from the optical systems is guided to the examinee's eye via the objective lens 22. Hereinafter, details of each optical system will be described.

### <Front Photographing Optical System>

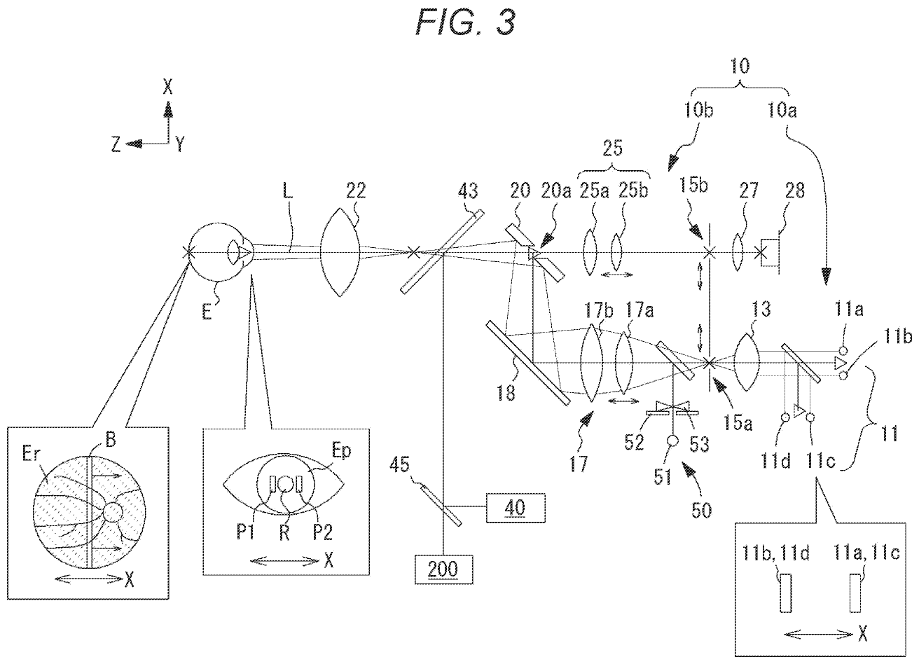

FIG. 3 is a schematic diagram of the front photographing optical system 10. Note that in FIG. 3, a pupil conjugate position conjugated with the pupil of the examinee's eye is indicated by a. “triangular mark” on the optical axis and a fundus conjugate position conjugated with the fundus of the examinee's eye is indicated by a “cross mark” on the optical axis.

The front photographing optical system has an irradiation optical system 10a and a light receiving optical system 10b. The irradiation optical system 10a has a light source unit 11, a lens 13, a slit-shaped member 15a, lenses 17a, 17b, a mirror 18, an apertured mirror 20, the objective lens 22, and the like. The light receiving optical system 10b has the objective lens 22, the apertured mirror 20, lenses 25a, 25b, a slit-shaped member 15b, an imaging element 28, etc.

The light source unit 11 has multiple types of light sources different from each other in a wavelength band. For example, the light source unit 11 has visible light sources 11a, 11b and infrared light sources 11c, 11d. As described above, two light sources are provided for each wavelength at the light source unit 11 of the present example. Two light sources with the same wavelength are arranged apart from the optical axis L on a pupil conjugate surface. Two light sources are next to each other along the X-direction which is a scanning direction in FIG. 3, and are arranged symmetrically with respect to the optical axis L. As shown in FIG. 3, the outer shapes of two light sources may be such a rectangular shape that a length in a direction crossing the scanning direction is longer than a length in the scanning direction.

Light from two light sources passes through the lens 13, and the slit-shaped member 15a is irradiated with such light. In the present example, the slit-shaped member 15a has a light transmitter (opening) formed elongated along the Y-direction. Thus, illuminating light is formed into a slit shape on a fundus conjugate surface (a region of the fundus Er illuminated in a slit shape is indicated by a reference character B).

The slit-shaped member 15a is displaced by the not-shown driver such that the light transmitter crosses the optical axis L in the X-direction. Thus, scanning with the illuminating light in the present example is implemented. Note that in the present example, scanning by the slit-shaped member 15b is also performed on a light receiving system side. In the present example, the light-projection-side and light-receiving-side slit-shaped members are driven in conjunction with each other by the single driver (actuator). Accordingly, a scanner including the slit-shaped members 15a, 15b is formed. The scanner may be, for example, an optical chopper. For details of an optical system employing the optical chopper, see “JP-A-2019-118721” filed by the present applicant, for example.

In the irradiation optical system 10a, the light from each light source is relayed by the optical system from the lens 13 to the objective lens 22, and accordingly, images of the light sources are formed on the pupil conjugate surface. That is, pupil images are, using two light sources, formed at positions apart from each other in the scanning direction on the pupil conjugate surface. In this manner, two light projection regions P1, P2 on the pupil conjugate surface are, in the present example, formed as images of two light sources.

The slit-shaped light having passed through the slit-shaped member 15a is relayed by the optical system from the lens 17a to the objective lens 22, and accordingly, an image is formed on the fundus Er. Thus, the illuminating light is formed into the slit shape on the fundus Er. The illuminating light is reflected on the fundus Er, and exits through the pupil Ep.

The apertured mirror 20 is an optical path coupler that couples an optical path of the irradiation optical system 10a and an optical path of the light receiving optical system 10b. The apertured mirror 20 reflects the illuminating light from the light source unit 11 to an examinee's eye E side, and causes part, which has passed through the opening, of fundus reflection light from the examinee's eye E to pass to an imaging element 28 side. Various beam splitters other than the apertured mirror 20 may be used as the optical path coupler. For example, instead of the apertured mirror 20, a mirror configured such that a light transmitter and a reflector are reversed from those of the configuration of the apertured mirror 20 may be used as the optical path coupler. Note that in this case, an independent optical path of the light receiving optical system 10b is on a reflection side of the mirror and an independent optical path of the irradiation optical system 10a is on a transmission side of the mirror. Each of the apertured mirror and mirrors as substitutes therefor can be further replaced with a combination of a half mirror and a light shield.

The opening of the apertured mirror 20 is at a position conjugated with the pupil of the examinee's eye. Thus, the fundus reflection light utilized for photographing is, on the pupil of the examinee's eye, limited to partial light passing through an image (pupil image) of the apertured mirror opening. The opening image on the pupil of the examinee's eye is a light receiving region R in the present example. The light receiving region R is formed between two light projection regions P1, P2 (images of two light sources). As a result of the image forming magnification of each image, the diameter of the opening, and an arrangement interval between two light sources being set as necessary, the light receiving region R and two light projection regions P1, P2 are formed so as not to overlap with each other on the pupil.

The fundus reflection light having passed through the objective lens 22 and the opening of the apertured mirror 20 forms an image of the slit-shaped region of the fundus Er at the fundus conjugate position via the lenses 25a, 25b. A light transmitter of the slit-shaped member 15b is arranged at such an image forming position, and therefore harmful light is removed.

The imaging element 28 is arranged at the fundus conjugate position. In the present example, a relay optical system 27 is provided between the slit-shaped member 15b and the imaging element 28. With this configuration, both the slit-shaped member 15b and the imaging element 28 are in a conjugate relation with the fundus. As a result, both harmful light removal and image formation are favorably performed. Instead, the relay optical system 27 between the imaging element 28 and the slit-shaped member 15b may be omitted, and both of these components may be arranged close to each other. In the present example, a device having a two-dimensional light receiving surface is used as the imaging element 28. This device may be, for example, a CMOS or a two-dimensional CCD. The image of the slit-shaped region of the fundus Er formed by the light transmitter of the slit-shaped member 15b is projected on the imaging element 28. The imaging element 28 has a sensitivity to both infrared light and visible light.

In the present example, an image (slit-shaped image) of a scanning position on the fundus Er is sequentially projected on each scanning line of the imaging element 28 as the fundus Er is scanned with the slit-shaped illuminating light. As described above, an entire image of a scanning area is projected on the imaging element 28 in a time-sharing manner. As a result, a front image (the two-dimensional reflection image) of the fundus is captured as the entire image of the scanning area.

Note that in the present example, the scanner in the light receiving optical system 10b is a device for mechanical scanning with slit-shaped light. On this point, the scanner is not limited to such a device. For example; the scanner on a light receiving optical system 10b side may be a device for electronic scanning with slit-shaped light. As one example, in a case where the imaging element 28 is the CMOS, scanning with slit-shaped light may be implemented by a rolling shutter function of the CMOS. In this case, an imaging surface region to be exposed with light is displaced in synchronization with the scanner in the irradiation optical system 10a, and therefore, photographing can be performed efficiently while the harmful light is removed. For example, a liquid crystal shutter may be used as the scanner for electronic scanning with the slit-shaped light.

The front photographing optical system 10 has a diopter corrector. In the present example, the diopter corrector (diopter correction optical systems 17, 25) is provided on each of the independent optical path of the irradiation optical system 10a and the independent optical path of the light receiving optical system 10b. Note that the diopter corrector may be provided on an optical path common between the irradiation optical system 10a and the light receiving optical system 10b.

Hereinafter, for the sake of convenience, the diopter correction optical system on an irradiation side will be referred to as an irradiation-side diopter correction optical system 17, and the diopter correction optical system on a light receiving side will be referred to as a light-receiving-side diopter correction optical system 25. The irradiation-side diopter correction optical system 17 of the present example includes the lens 17a, the lens 17b, and the driver (not shown). The light-receiving-side diopter correction optical system 25 of the present example includes the lens 25a, the lens 25b, and the driver (not shown). In the irradiation-side diopter correction optical system 17, an interval between the lens 17a and the lens 17h is changed. In the light-receiving-side diopter correction optical system 25, an interval between the lens 25a and the lens 25b is changed. With this configuration, diopter correction is performed in each of the irradiation optical system 10a and the light receiving optical system 10b.

The front photographing optical system 10 further has a target projecting optical system 50. The target projecting optical system 50 projects, as focus targets, two split targets on the fundus Er. The split targets are utilized for focus detection.

For example, the target projecting optical system 50 may have at least an infrared light source 51, a target plate 52, and a declination prism 53. In the present example, the target plate 52 is arranged at a position corresponding to the imaging surface in the light receiving optical system 10b. Similarly, the target plate 52 is also arranged at a position corresponding to each of the slit-shaped members 15a, 15b. Specifically, in, e.g., a case where: a diopter correction amount is 0 D on the irradiation side and the light receiving side, the target plate 52 is arranged at a position substantially conjugated with the fundus Er of an emmetropic eye (a 0D eye). The declination prism 53 is, on an examinee's eye side with respect to the target plate 52, arranged close to the target plate 52.

For example, the target plate 52 forms the slit-shaped light into the targets. The declination prism 53 separates a target light flux having passed through the target plate 52, thereby forming the split targets. The separated split targets are projected on the fundus Er by way of the irradiation-side diopter correction optical system 17 to the objective lens 22. Thus, the split targets are on the fundus image (e.g., the fundus observation image). Note that in the present example, one of two separated split targets passes through the light projection region P1, and reaches the fundus Er of the examinee's eye. The other split target passes through the light projection region P2, and reaches the fundus Er of the examinee's eye.

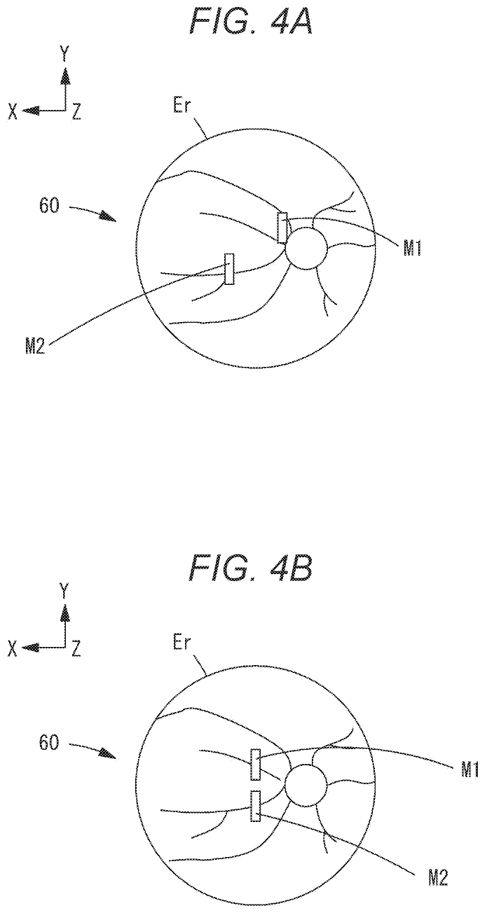

FIGS. 4A and 4B show, as an example, a fundus observation image 60, split targets M1, M2 being on the fundus observation image GO. In the present example, the split target M1 passes through the light projection region P1, and is projected on the fundus Er. The split target M2 passes through the light projection region P2, and is projected on the fundus Er. FIG. 4A shows the image in a case where a focus state is not properly adjusted and the target plate 52 is shifted from the fundus conjugate position. In this case, two split targets M1, M2 are at positions shifted from each other in the X-direction. FIG. 4B shows the image in a case where the focus state is properly adjusted and the target plate 52 is arranged at the fundus conjugate position. In this case, two split targets M1, M2 are at positions coincident with each other in the X-direction. In the present example, a conjugate relation between the fundus Er and the target plate 52 is adjusted by the irradiation-side diopter correction optical system 17 arranged between the declination prism 53 and the fundus Er. Thus, in the present example, defocusing is performed while the irradiation-side diopter correction amount and the light-receiving-side diopter correction amount are being adjusted. Each of the irradiation-side and light-receiving-side diopter correction amounts is adjusted such that two split targets are coincident with each other, and in this manner, each of the imaging surface and the slit-shaped members 15a, 15b is in a conjugate positional relation with the fundus Er.

For example, the infrared wavelength of the infrared light source (the infrared light sources 11c, 11d) in the irradiation optical system 10a and the infrared wavelength of the infrared light source (the infrared light source 51) in the target projecting optical system 50 may be the same as each other. With this configuration, the fundus reflection light obtained by the irradiation optical system 10a and the fundus reflection light obtained by the target projecting optical system 50 are, in the present example, imaged by the single imaging element 28 so that the fundus observation image including the split targets can be obtained. Needless to say, the infrared wavelength may be different between the infrared light sources. In this case, e.g., an imaging element having a sensitivity to a predetermined infrared wavelength range may be used.

### <Anterior Segment Observing Optical System>

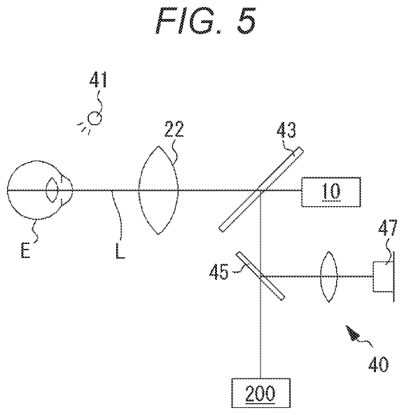

FIG. 5 is a schematic diagram of the anterior segment observing optical system 40.

The anterior segment observing optical system 40 images the anterior segment of the examinee's eye E, thereby acquiring an anterior segment observation image. The anterior segment observing optical system 40 illuminates the anterior segment with infrared light, thereby photographing a front image of the anterior segment. The anterior segment observing optical system has a light source 41, the half mirror 45, an imaging element 47, the dichroic mirror 43, the objective lens 22, and the like. For example, the light source 41 is an infrared light source, and illuminates the examinee's eye E. For example, the imaging element 47 is a two-dimensional imaging element, and is arranged at a position optically conjugated with the pupil Ep. The dichroic mirror 43 and the objective lens 22 are shared with the front photographing optical system. Note that the anterior segment observing optical system 40 may be configured such that the anterior segment is imaged via an optical path independent from other optical systems.

### <OCT Optical System>

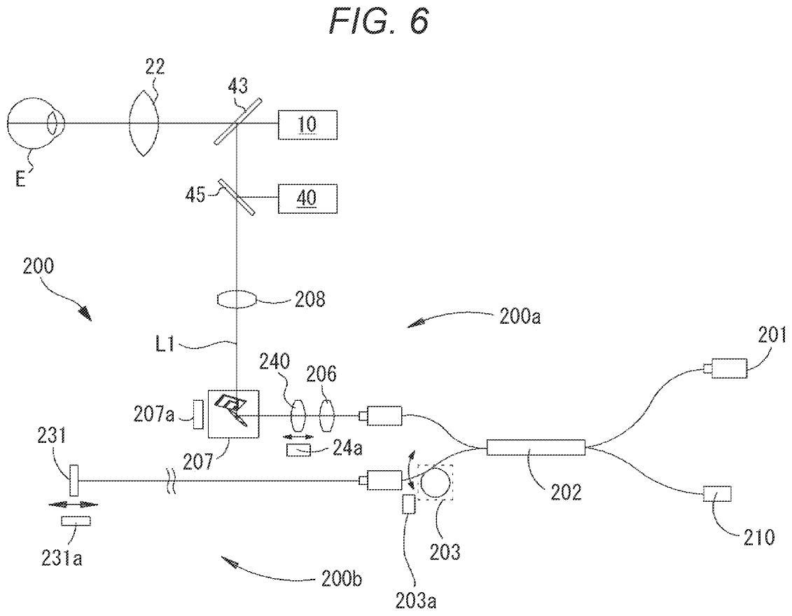

FIG. 6 shows a schematic configuration of the OCT optical system 200. As one example, the OCT optical system 200 as a SD-OCT optical system will be described.

The OCT optical system 200 obtains, by imaging, OCT data on the fundus Er. The OCT optical system 200 includes an OCT light source 201, a coupler (light splitter) 202, a polarizer 203, a measuring optical system 200a, a reference optical system 200b, and a detector 210.

In SD-OCT, a wideband light source is utilized as the OCT light source 201. Light from the OCT light source 201 is divided into measurement light (sample light) and reference light by the coupler 202. The measurement light is guided to the fundus Er via the measuring optical system 200a. The reference light is guided to the reference optical system 200b.

In the present example, the measuring optical system 200a has a collimator lens 206, a focus lens 240, a scanner 207, a lens 208, and the objective lens 22.

The measurement light is guided to the scanner 207 via the collimator lens 206 and the focus lens 240. The scanner 207 two-dimensionally scans the fundus Er with the measurement light. The scanner 207 is arranged at a position substantially conjugated with the pupil of the examinee's eye E. Thus, the measurement light pivots about the pupil of the examinee's eye E. In the present example, two galvanometer mirrors are used as the scanner 207, for example. The fundus Er is, via the objective lens 22, irradiated with the measurement light having passed through the scanner 207. The measurement light from the fundus Er passes through the measuring optical system 200a in reverse order, and is guided to the detector 210.

In the present example, the reference optical system 200b is a reflective optical system, and mainly includes a reference mirror 231. The reference light makes one round trip between the coupler 202 and the reference mirror 231. The reference light having entered the coupler 202 after one round trip is guided to the detector 210.

In the present example, the reference mirror 231 is movable in an optical axis direction by a driver 231a. The optical path length of the reference optical system 200b is changed according to the position of the reference mirror 231. As a result, a difference in an optical path length between the measurement light and the reference light is adjusted.

In the present example, the case where the reference optical system 200b is the reflective optical system has been described. On this point, the reference optical system 200b may be a transmission optical system (e.g., an optical fiber).

In the present example, the polarizer 203 is arranged between the coupler 202 and the reference optical system 200b. The polarizer 203 adjusts a polarization state of the reference light. The polarizer 203 is driven by a driver 203a, thereby changing the polarization state of the reference light. Note that arrangement of the polarizer 203 is not limited to that in the example of FIG. 6 and the polarizer 203 may be arranged at a position for adjusting a polarization state of the measurement light.

The detector 210 receives interference light of the reference light and the return measurement light from the fundus Er. In SD-OCT, a spectrometer is utilized as the detector 210. The OCT data on the fundus Er is generated based on a spectral interference signal from the detector 210.

Generally, a pupil diameter required for the OCT optical system can be a sufficiently-smaller value than a pupil diameter required for an observing optical system in which pupil division is set spatially. In the present example, projection and reception of the measurement light between the OCT optical system 200 and the examinee's eye E are performed inside the light receiving region R on the pupil of the examinee's eye E. Thus, in the present example, as long as a favorable fundus observation image is acquired, it is guaranteed that projection and reception of the measurement light are favorably performed at least on the pupil of the examinee's eye E.

### <Controller>

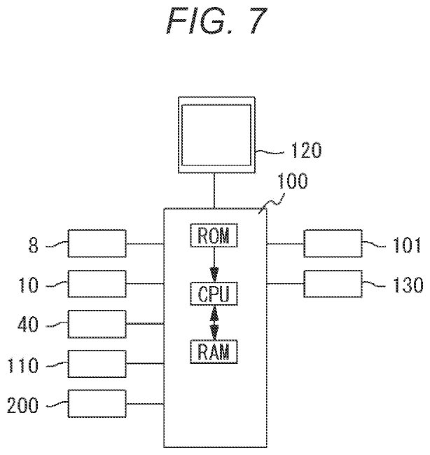

FIG. 7 shows a control system of the fundus photographing apparatus 1. The fundus photographing apparatus 1 has the controller 100. The controller 100 is a processing apparatus (processor) that performs control and calculation for each component. The controller 100 includes a CPU, a RAM, a ROM, etc. For the sake of convenience, the controller 100 processes various images obtained by the fundus photographing apparatus 1. In other words, the controller 100 also serves an image processor.

The controller 100 is electrically connected to each of the driver 8, the front photographing optical system 10, the anterior segment observing optical system 40, the face photographing camera 110, the OCT optical system 200, the monitor 120, an input interface 130, a storage 101, and the like.

The controller 100 controls each of the above-described components based on an operation signal output from the input interface 130. The input interface 130 is an operation inputter that receives examiner's operation. The input interface 130 may be, for example, a mouse or a keyboard.

The storage 101 may be a non-transitory storage medium capable of holding stored contents even after a power supply is cut off. For example, the storage 101 may be a hard disk drive, a flash ROM, a USB memory, and the like. For example, the storage 101 stores various control programs, various types of fixed data, and the like. Moreover, the storage 101 stores photographic images obtained by the fundus photographing apparatus″, for example. The photographic images may be stored in an external storage apparatus a storage apparatus connected to the controller 100 via a LAN and a WAN).

### <Description of Operation>

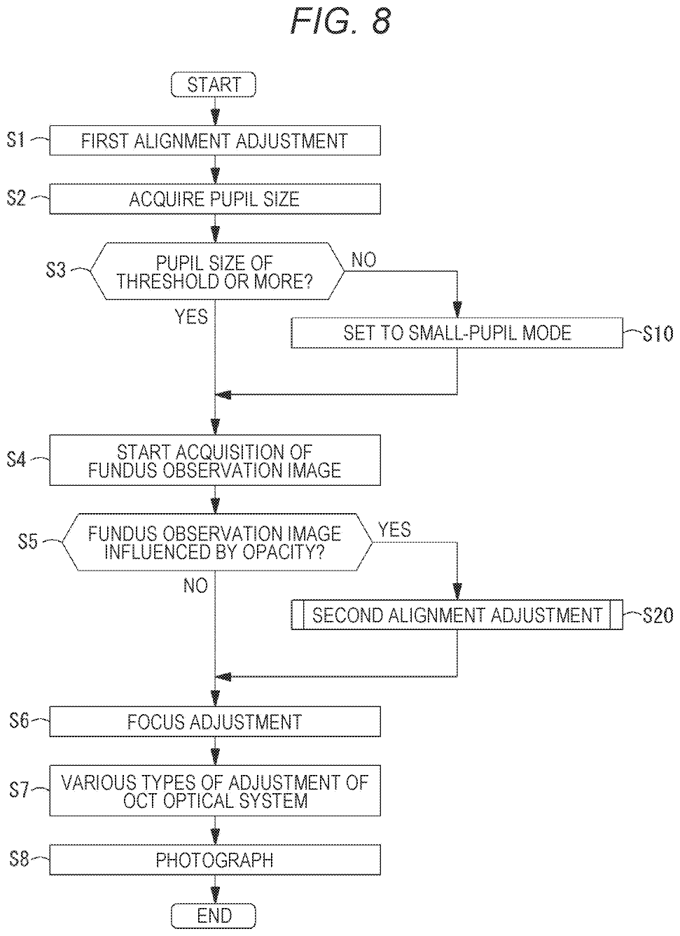

Next, operation of the fundus photographing apparatus 1 in the present example will be described with reference to FIGS. 8 to 13.

In the present example, an alignment state is automatically adjusted so that the two-dimensional reflection image (the color fundus image) of the fundus Er and the OCT data on the fundus Er can be obtained so as to avoid opacity even in a case where there is the opacity in the optic media of the examinee's eye E. In the present example, the case of performing automatic alignment will be described as one example.

FIG. 8 is a flowchart showing the flow of operation in the fundus photographing apparatus 1.

Operation is started with the face of the examinee arranged on the face supporting unit 9. First, the photographing unit 3 is adjusted relative to the examinee's eye E such that the position thereof reaches such a position that the fundus observation image can be acquired. The position of the photographing unit 3 is adjusted based on the facial image acquired via the face photographing camera 110 and the anterior segment observation image acquired via the anterior segment observing optical system 40.

For example, the controller 100 acquires the facial image via the face photographing camera 110. The controller 100 detects the position of at least any of the right and left examinee's eyes included in the facial image. The controller 100 adjusts, based on the detected positional information, the position of the photographing unit 3 to such a position that the anterior segment can be observed.

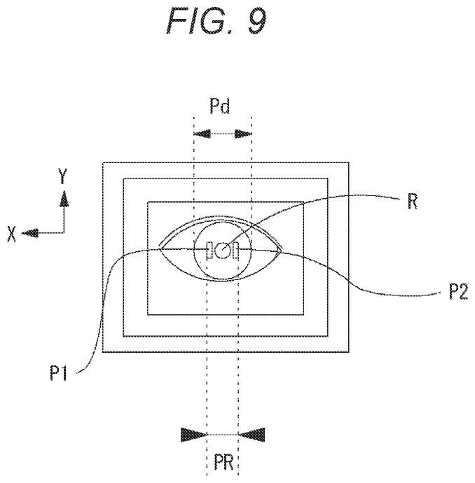

After alignment adjustment has been performed based on the facial image, the anterior segment observation image as shown in FIG. 9 is acquired via the anterior segment observing optical system 40. Note that in FIG. 9, the light projection regions P1, P2 and the light receiving region R are merely shown for the sake of convenience in description. The controller 100 adjusts the positional relation between the examinee's eye E and the photographing unit 3 based on the anterior segment observation image. In the present example, the controller 100 sets an alignment reference position, targeting such a positional relation that the pupil center and the image center (the position of the photographing optical axis L in the present example) are substantially coincident with each other. Misalignment from the reference position is detected, and the photographing unit 3 is moved in such a direction that the misalignment in the X- and Y-directions is eliminated. The misalignment may be detected as a shift amount between the pupil center and the photographing optical axis on the anterior segment observation image. For example, in a case where the fundus photographing apparatus 1 has an alignment projecting optical system that projects an alignment target onto the corneal apex, the misalignment may be detected as a shift amount between the alignment target and the photographing optical axis.

As described above, in the present example, the positional relation in the X- and Y-directions between the examinee's eye E and the photographing unit 3 is, as a result of first alignment adjustment, adjusted such that the center of the light receiving region R (i.e., the photographing optical axis) is coincident with the pupil center. Note that if the reference in the first alignment adjustment is set to the corneal apex, the above-described positional relation may be adjusted such that the center of the light receiving region R is coincident with the corneal apex.

The controller 100 also adjusts the positional relation in the Z-direction between the examinee's eye E and the photographing unit 3 such that an interval between the examinee's eye E and the photographing unit 3 reaches a predetermined distance. For example, the photographing unit 3 may be moved in the front-back direction such that the focus of the anterior segment observation image is adjusted to the pupil Ep. Using various alignment targets, the controller 100 may adjust the positional relation in the Z-direction between the examinee's eye E and the photographing unit 3.

Next, the controller 100 acquires a pupil diameter Pd (see FIG. 9) as a pupil size (pupil information in the present example) based on the anterior segment observation image, and compares the pupil diameter Pd with a threshold. For example, the controller 100 detects a pupil region from the anterior segment observation image by means of a technique such as edge detection, and acquires the pupil diameter Pd. The pupil diameter Pd may be acquired as a measurement result from an apparatus different from the fundus photographing apparatus 1, or may be manually input by the examiner.

The controller 100 compares the acquired pupil diameter Pd with the threshold. As one example, the threshold may be substantially at the same level as the entire width PR (a width in the X-direction, see FIG. 9) of a region including the light projection regions P1, P2 and the light receiving region R.

In a case where the pupil diameter Pd is the threshold or more, acquisition of the fundus observation image is started (S4).

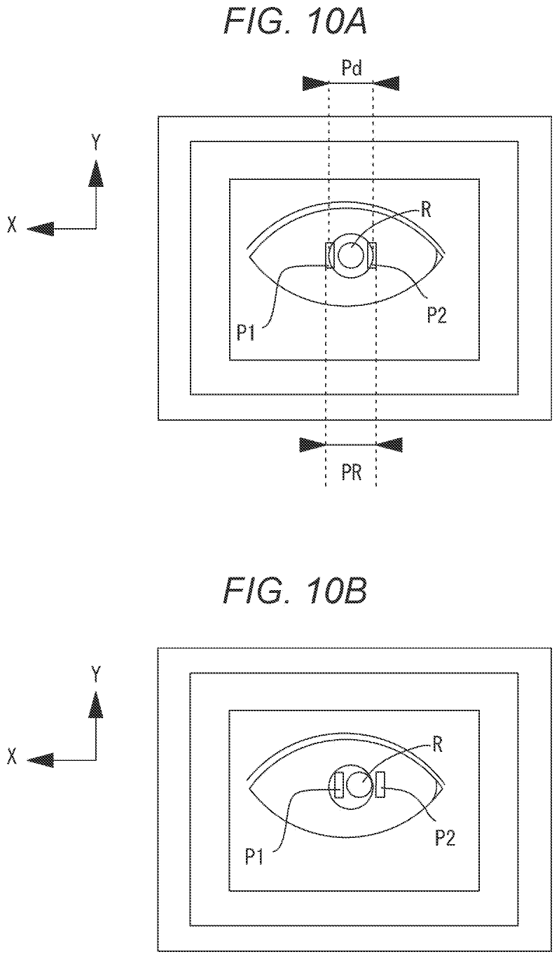

FIGS. 10A and 10B show anterior segment observation images acquired in a case where the pupil diameter Pd is less than the threshold. As shown in FIG. 10A, in a case where the pupil diameter Pd is less than the threshold (S3: No), the light projected via the light projection regions P1, P2 is eclipsed by iris in the present example. In this case, the fundus Er cannot be properly photographed. For this reason, in this case, a small-pupil mode is set (S10), and acquisition of the fundus observation image in the small-pupil mode is started (S4).

As shown in FIG. 10B, in the small-pupil mode of the present example, the alignment reference position is offset (eccentric) in the X-direction such that one of the light projection regions P1, P2 is, together with the light receiving region R, arranged in the pupil region preferentially over the other one of the light projection regions P1, P2. The positional relation between the examinee's eye E and the photographing unit 3 is adjusted according to the offset of the reference position. Note that the amount of offset of the reference position may be set according to the pupil diameter Pd or a fixed value. As shown in FIG. 10B, the light projection region P1 is preferentially arranged in the present example. In the small-pupil mode, the controller 100 may turn on only one, which forms the light projection region P1, of two infrared light sources 11c, 11d for observation, and may turn off the other one of two infrared light sources 11c, 11d.

After acquisition of the fundus observation image has been started (S4), the controller 100 determines the presence or absence of influence of the opacity of the optic media on the fundus observation image (i.e., whether or not the influence is at an acceptable level) (S5).

In the present example, the presence or absence of the influence of the opacity is determined utilizing the split targets M1, M2. The controller 100 causes the target projecting optical system 50 to project the split targets.

As described above, in the present example, two split targets are projected and received via the light projection regions P1, P2 and the light receiving region R. Thus, it is assumed that the split targets with which the inside of the pupil region is irradiated without the split targets eclipsed by iris are on the fundus observation image as long as the projected and received light is not blocked by the opacity. That is, it is assumed that two split targets M1, M2 are on the image in the case of a sufficient pupil size and at least one of the split targets M1, M2 is on the image in the case of the small-pupil mode. The controller 100 detects these split targets, and based on a detection result, determines the presence or absence of the influence of the opacity of the optic media.

For example, in a case where one or two intended target images are not properly detected, it may be determined that there is the influence of the opacity of the optic media on the fundus observation image. In a case where the luminance of the detected split target is lower than a threshold, it may be determined that there is the influence of the opacity of the optic media on the fundus observation image. In a case where it is determined that there is the influence of the opacity of the optic media on the fundus Observation image (S5: Yes), second alignment adjustment (S20) is executed, and alignment adjustment for avoiding the opacity is further performed. Details of the second alignment adjustment (S20) will be described later.

In a case where it is determined that there is no influence of the opacity of the optic media on the fundus observation image (S5: No), focus adjustment is executed (S6) after the second alignment adjustment (S20) has been executed.

For example, the controller 100 detects a separation state of the split targets M1, M2 from the fundus Observation image, and drives the diopter correctors (the diopter correction optical systems 17, 25) to defocus the front photographing optical system 10 such that the split targets M1, M2 are coincident with each other. The controller 100 drives the focus lens 240 of the OCT optical system 200 in conjunction with the diopter correctors (the diopter correction optical systems 17, 25) of the front photographing optical system 10. Accordingly; focus adjustment for the OCT optical system 200 is performed.

Note that in the case of the small-pupil mode, only at least one of the split targets M1, M2 is on the fundus observation image. Thus, focus adjustment as described above is performed such that an intended one of two targets M1, M2 is arranged at a preset matching position. The matching position described herein may be, for example, a matching position in a case where it is assumed that two split targets M1, M2 are on the fundus observation image.

Next, various types of adjustment for the OCT optical system 200 are performed (S7). The controller 100 performs fine focus adjustment, optical path length adjustment, polarization state adjustment (polarizer adjustment), and the like while acquiring the OCT data via the OCT optical system 200. For details of adjustment, see JP-A-2015-195876 filed by the present applicant, for example. As a result of adjustment for the OCT optical system 200, the OCT data on the fundus Er can be acquired with a high sensitivity and a high resolution.

Next, the controller 100 executes photographing (S8). For example, the controller 100 controls the OCT optical system 200 to obtain the OCT data by photographing. Thereafter, the controller 100 controls the front photographing optical system 10 to photograph the color fundus image. The controller 100 may execute each type of photographing based on operation input which is a trigger for photographing, or may automatically execute each type of photographing. A photographing result is stored in the storage 101. The photographing result may be displayed on the monitor 120.

### <Second Alignment Adjustment>

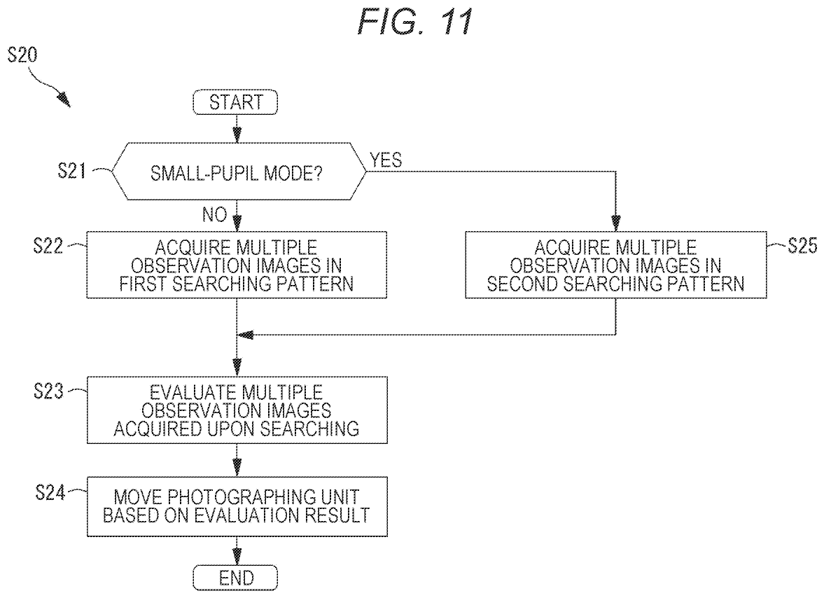

Next, details of the second alignment adjustment will be described with reference to a flowchart of FIG. 11.

In the second alignment adjustment, the controller 100 searches such an alignment state that one or two intended target images can be favorably detected while causing the positional relation between the examinee's eye E and the photographing unit 3 to transition (change).

In the present example, it is assumed that for the fundus observation image acquired substantially in real time, the split targets are detected as needed during searching of the alignment state and the influence of the opacity of the optic media is evaluated by a technique similar to that of S5.

In the present example, a positional relation transition pattern (also referred to as a searching pattern) varies according to whether or not the small-pupil mode is set.

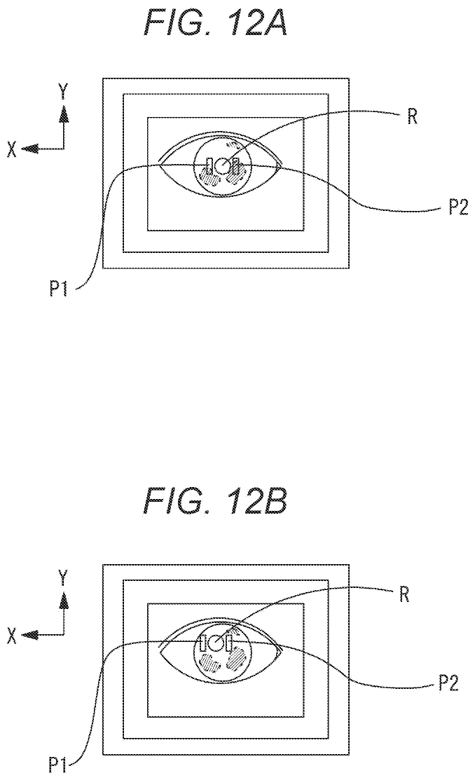

First, a first searching pattern to be executed in a normal state (the case of S21: No) will be described with reference to FIGS. 12A and 12B. In this case, such an alignment state that two split targets M1, M2 are both detected is searched.

The controller 100 moves the photographing unit 3 within such an area that the light projection regions P1 P2 and the light receiving region R are not outside the pupil. The movement area may be, for example, set based on the pupil diameter I'd.

In the first searching pattern, the controller 100 randomly moves the photographing unit 3 in the X- and Y-directions within such an area that the light projection regions P1, P2 and the light receiving region R are not outside the pupil (FIG. 12A to FIG. 12B), and acquires the fundus observation image at each position. Note that the first searching pattern is not necessarily the random pattern and may be a preset pattern. For example, the first searching pattern may be such a pattern that the photographing unit 3 is first moved in the Y-direction and is subsequently moved in the X-direction, or may be other patterns.

Based on the observation images obtained during searching, the controller 100 may determine, as needed, the direction and amount of movement of the photographing unit 3. For example, in some cases, only one split target M1, M2 is detected during searching although the inside of the pupil region is irradiated with two split targets M1, M2. In this case, it is estimated that there is no opacity in one, which corresponds to the detected target image, of the light projection regions P1, P2 and the light receiving region R. Using this fact, positional relation candidates having a probability of the fundus observation image being favorably acquired are narrowed down during searching, and at the same time, transition to the narrowed-down positional relation may be made.

During searching, the controller 100 acquires, in association with each observation image, information indicating the alignment state when such an observation image is acquired. The controller evaluates the influence of the opacity on each observation image (S23), and changes the alignment reference position with reference to the alignment state based on the observation image with no (less) influence of the opacity. Further, the controller 100 adjusts the positional relation between the examinee's eye E and the photographing unit 3 according to the changed alignment reference position (S24).

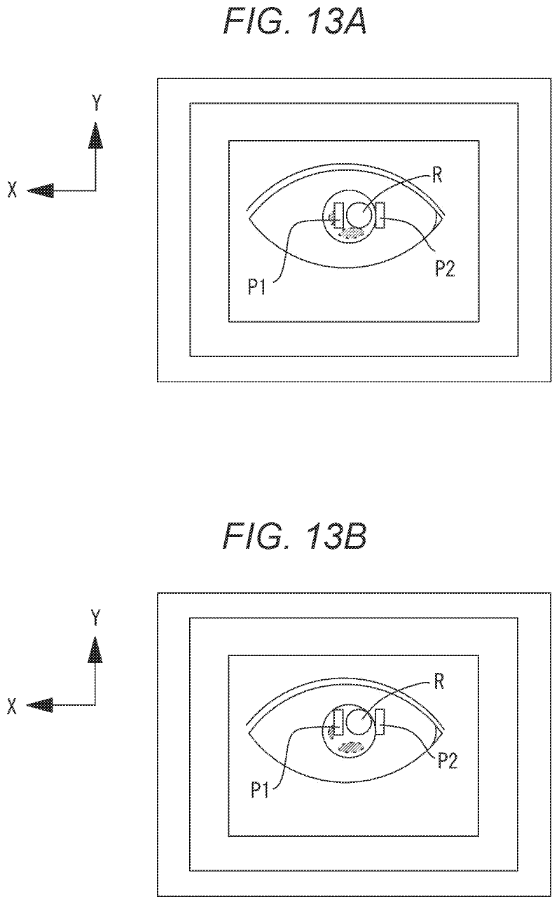

Next, a second searching pattern to be executed in the small-pupil mode (the case of S21: Yes) will be described with reference to FIGS. 13A and 13B, For example, in a case where the light projection region P1 is preferentially arranged in the pupil region, such an alignment state that the split target M1 projected and received via the light projection region P1 and the light receiving region R is properly detected is searched.

In this case, the controller 100 conduct a search while moving the photographing unit 3 in the Y-direction mainly under a condition where the light projection region P1 and the light receiving region R are not outside the pupil (FIG. 13A to FIG. 13B). That is, the photographing unit 3 is preferably preferentially roved in the Y-direction of the X- and Y-directions. In the present example, the light projection region P1 and the light receiving region R are arranged next to each other in the X-direction. Thus, in the case of an eye with a small pupil, it is assumed that a space for movement in the X-direction is difficult to be ensured within the pupil region.

Note that transition of the positional relation between the examinee's eye F and the photographing unit 3 in the second searching pattern may be, as in the first searching pattern, random, preset, or determined sequentially during searching.

The controller 100 may switch, in the middle of searching, the light projection region preferentially arranged in the pupil region between two light projection regions P1, P2, for example. An eccentric state in which the light projection region P1 is included in the pupil region as in FIG. 10B may be switched to an eccentric state in which the light projection region P2 is included in the pupil region. Accordingly, the probability of a favorable fundus observation image being acquired increases.

In the present example, adjustment to such an alignment state that the fundus observation image can be properly acquired even with the opaque optic media of the examinee's eye is performed as described above.

As described above, in the present example, focus adjustment is performed thereafter. As a result of alignment adjustment, focus adjustment is performed with the split targets favorably on the fundus observation image. Thus, in the present example, focus adjustment is less likely to be failed.

In the present example, the fundus observation image and the color fundus image are captured by the same optical system. Thus, in this alignment state, the color fundus image is favorably photographed. Moreover, in the present example, projection and reception of the measurement light between the OCT optical system 200 and the examinee's eye E are performed inside the light receiving region R on the pupil of the examinee's eye E. Thus, the OCT data can also be favorably obtained.

In the present example, the front photographing optical system 10 is a slit scan type optical system, and the harmful light is removed by the slit-shaped member 15. Thus, even in a case where the optical axis of the front photographing optical system 10 displaces from the center of the anterior segment in order to reduce the influence of the opacity, noise light from the optic media is suitably removed by the slit-shaped member 15. Thus, a favorable color fundus image can be photographed.

### [Modifications]

The techniques disclosed above in the embodiments are merely examples. Thus, the techniques disclosed above in the embodiments can also be changed. For example, only some of the multiple techniques described above as examples in the embodiments may be executed.

For example, in the first example, the case where such a positional relation that two split targets M1, M2 are both properly detected is searched has been described. Note that the present disclosure is not limited to above and such a positional relation that one of two split targets M1, M2 is properly detected may be searched. In this case, proper alignment adjustment is easily performed for an examinee's eye with a high degree of opacity.

In the above-described example, the case where focus adjustment is performed after alignment adjustment has been described. On this point, alignment adjustment and focus adjustment may be performed in parallel. That is, while the positional relation between the examinee's eye and the photographing unit is being changed based on the fundus observation image, focus adjustment may be started when at least one of the split targets M1, M2 is detected. In this case, a time required for various types of adjustment until photographing is shortened.

In the above-described example, when alignment based on the anterior segment observation image is completed, the examiner may select, via the input interface 130, whether or not the small-pupil mode is to be set. In this case, the anterior segment observation image of the examinee's eye may be displayed on the monitor 120, and the examiner may check a positional relation between the pupil of the examinee's eye and each of the light projection region and the light receiving region, for example.

For example, in a case where multiple fundus observation images are acquired at positions different from each other in the X- and Y-directions, transition of the positional relation between the examinee's eye and the photographing unit may be made based on examiner's operation. For example, the examiner may operate a joystick to adjust the positional relation. Further, the controller 100 may limit an area where transition can be made in the X- and Y-directions, considering the pupil information. The controller 100 may control, regardless of examiner's operation, the positional relation such that the pupil image obtained using the optical system is not outside the pupil.

In the above-described example, the case where the split targets are detected as a result of searching has been described. However, a case where no split targets are detected even after searching is assumed. In this case, the controller 100 may execute photographing in a state in which the alignment reference position based on alignment on the basis of the anterior segment observation image is kept set.

The front photographing optical system 10 of the above-described example is the slit scan type optical system, and all of the light receiving region R and two light projection regions P1, P2 are formed in line. On the other hand, in a spot scan type optical system, a light projection region and a light receiving region may be formed in line, for example. Further, in addition to the light projection region and the light receiving region formed in line, at least one or more light projection regions and one or more light receiving, regions arranged in a direction crossing such a line may be further formed.

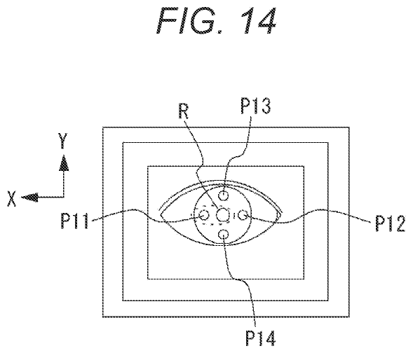

For example, FIG. 14 shows, on the anterior segment observation image, pupil images (each region (each pupil image) P11, P12, P13, P14, R) in an optical system in which the light receiving region R and the light projection regions P11, P12 are formed in line in the X-direction and the light receiving region R and the light projection regions P13, P14 are formed in line in the Y-direction. The illuminating light may be irradiated simultaneously or selectively via the multiple light projection regions P11, P12, P13, P14.

In such an apparatus, in the case of the second alignment mode, the controller selects any one of the multiple light projection regions P11, P12, P13, P14, and the direction of arrangement of the selected light projection region and the light receiving region R is taken as a first direction. The controller 100 displaces the positional relation between the examinee's eye and the photographing unit at least in a direction crossing the first direction from a predetermined positional relation guided in the first alignment. For example, in a case where the light projection region P11 has been selected, the positional relation between the examinee's eye and the photographing unit is displaced at least in the Y-direction. In this manner, guide to the second alignment state may be performed.

Note that in the example of FIG. 14, the multiple (four in the figure) light projection regions are formed for the single light receiving region R. On this point, the number of light receiving regions and the number of light projection regions can be changed as necessary.

The foregoing detailed description has been presented for the purposes of illustration and description. Many modifications and variations are possible in light of the above teaching. It is not intended to be exhaustive or to limit the subject matter described herein to the precise form disclosed. Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims appended hereto.

What is claimed is:

1. A fundus photographing apparatus comprising:
a photographing unit including a front photographing optical system that forms, on a pupil of an examinee's eye, an illuminating light projection region and an illuminating light receiving region next to each other in a first direction, the front photographing optical system scanning a fundus of the examinee's eye with illuminating light to acquire a two-dimensional reflection image of the fundus;
a driver that moves the photographing unit relative to the examinee's eye; and
a processor that switches, between a first alignment mode and a second alignment mode, a control of guiding a positional relation between the examinee's eye and the photographing unit,
the positional relation being guided to a first alignment state in a predetermined positional relationship in the first alignment mode, and
being guided to a second alignment state displaced at least in a direction crossing the first direction from the first alignment state in the second alignment mode.

2. The fundus photographing apparatus according to claim 1, wherein
the light projection region and the light receiving region formed next to each other in the first direction are non-concentrically arranged in line.

3. The fundus photographing apparatus according to claim 1, wherein
the processor acquires opacity information which is information regarding opacity in optic media of the examinee's eye, and based on the opacity information, selectively sets any of the first alignment mode and the second alignment mode.

4. The fundus photographing apparatus according to claim 1, wherein
the processor acquires pupil information as information regarding a pupil region of the examinee's eye, and guides the positional relation in consideration of the pupil information.

5. The fundus photographing apparatus according to claim 4, wherein
the processor acquires, as the pupil information, information regarding a pupil size of the examinee's eye, and according to the pupil size, changes the control of guiding the positional relation based on a fundus observation image which is the two-dimensional reflection image.

6. The fundus photographing apparatus according to claim 1, wherein
the photographing unit further includes an OCT optical system that obtains, by photographing, OCT data on the fundus based on an optical interference principle, and
the processor obtains, by photographing, the OCT data on the fundus and the two-dimensional reflection image of the fundus in the second alignment state in the second alignment mode.

7. The fundus photographing apparatus according to claim 1, further comprising:
an observing optical system that projects observation light which is infrared light onto the fundus of the examinee's eye and receives the observation light from the examinee's eye to acquire a fundus observation image which is a two-dimensional reflection image based on the observation light,
wherein the processor guides, based on the fundus observation image, the positional relation between the examinee's eye and the photographing unit to the second alignment state in the second alignment mode.

8. The fundus photographing apparatus according to claim 7, wherein
the photographing unit further includes a target projecting optical system that projects, onto the fundus, a target light flux for adjusting a photographing condition for the front photographing optical system, and
the processor detects a target image formed on the fundus observation image based on the target light flux, and guides the positional relation based on the target image.
